SEQUENCE LISTING (2 sequences)

SEQ ID NO: 1 - protein, 317 aa, Homo sapiens
  source 1..317: note = SUPER-REPRESSOR- IkB(srIkB)
MFQAAERPQEWAMEGPRDGLKKERLLDDRHDAGLDAMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 2 - protein, 317 aa, Homo sapiens
  source 1..317: note = IkB-a
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

(19) United States
(12) Patent Application Publication
(10) Pub. No.: US 2025/0360182 A1
(43) Pub. Date: Nov. 27, 2025

(54) THERAPEUTIC USE OF EXOSOME CONTAINING SUPER REPRESSOR- I¿B(SRI¿B) FOR LIVER DISEASE

(71) Applicant: ILIAS BIOLOGICS INC, Daejeon (KR)

(72) Inventors: Chulhee CHOI, Seoul (KR); Jaekwang YOO, Seoul (KR); Byung-Koo OH, Seoul (KR)

(21) Appl. No.: 18/875,118

(22) PCT Filed: Jun. 16, 2023
(86) PCT No.: PCT/KR2023/008367

§ 371 Date: Dec. 13, 2024

(30) Foreign Application Priority Data
Jun. 17, 2022 (KR) 10-2022-0073901
Jan. 6, 2023 (KR) 10-2023-0002047

(51) Int. Cl. A61K 38/16; A61K 9/127; A61P 1/16

(52) U.S. Cl. CPC A61K 38/16; A61K 9/127; A61P 1/16

(57) ABSTRACT

The present invention relates to the therapeutic use of exosome comprising super-repressor-IκB (srIκB) for liver disease.

### CROSS-REFERENCE TO RELATED APPLICATIONS

The present application is the 35 U.S.C. § 371 national phase of International Application No. PCT/KR2023/008367, filed on Jun. 16, 2023, which claims the benefit of Korean Patent Application Nos. 10-2022-0073901, filed on Jun. 17, 2022, and 10-2023-0002047, filed Jan. 6, 2023, all of which applications are incorporated herein by reference in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 13, 2024, is named “059520.00040.xml” and is 3,821 bytes in size. The sequence listing contained in this .XML file is part of the specification and is hereby incorporated by reference herein in its entirety.

### TECHNICAL FIELD

The present invention relates to the therapeutic use of an exosome containing super-repressor-IκB (srIκB) for liver disease.

### BACKGROUND ART

Liver diseases vary in type and severity, ranging from fatty liver to cirrhosis, and are often not discovered until they are in advanced stages because they do not have any initial subjective symptoms. The causes of liver disease are diverse, such as infection by viruses or bacteria, alcohol or toxic substances, excessive accumulation of fat or heavy metals, and abnormal immune responses, etc. Although various treatments are used for liver diseases depending on the cause and type of disease, there are many cases where conventionally known therapeutic agents do not respond, and thus they account for the leading cause of death worldwide. Accordingly, there is still a need to develop drugs for treating liver disease. Meanwhile, exosomes have recently attracted considerable attention as novel bio-carriers for gene/drug delivery. Exosomes are extracellular vesicles (EVs) that play an important role in intercellular communication by delivering bioactive substances to recipient cell or affecting the signaling pathways of target cell.

The present inventors have developed exosomes containing physiologically active substances capable of preventing or treating liver diseases, and confirmed that the exosomes stably and effectively deliver physiologically active substances to liver cell and tissues, thereby providing excellent treatment effects for liver disease, and accordingly, it is intended to provide the exosome of the present invention as a preventive or therapeutic agent for liver disease.

### DISCLOSURE

### Technical Problem

The present invention is to provide a pharmaceutical composition for preventing or treating liver disease, including an exosome containing super-repressor-IκB (srIκB) as an active ingredient.

### Technical Solution

One aspect for implementing the present invention relates to a pharmaceutical composition for preventing or treating liver disease, including an exosome containing super-repressor-IκB (srIκB) as an active ingredient.

Another aspect for implementing the present invention relates to a method for preventing or treating liver disease, including treating an exosome containing super-repressor-IκB (srIκB) protein to an individual at risk of liver disease.

### Advantageous Effects

The exosome containing super-repressor-IκB (srIκB) according to the present invention can be effectively used for the prevention or treatment of liver disease.

### BRIEF DESCRIPTION OF DRAWINGS

FIG. 1 is a schematic view of the production of a biliary stricture-induced animal model according to one embodiment of the present invention.

FIG. 2 is a diagram showing the measurement of the relative weight (%) of the liver when the vehicle and Exo-srIκB were administered to the biliary stricture-induced animal model according to one embodiment of the present invention. In particular, FIG. 2b shows the measurement of the relative weight (%) of the liver according to the body weight (g) of the animal.

FIG. 3 is a diagram confirming liver abscess when the vehicle and Exo-srIκB were administered to the biliary stricture-induced animal model according to one embodiment of the present invention.

FIG. 4 is a diagram confirming the symptoms of jaundice when the vehicle and Exo-srIκB were administered to the biliary stricture-induced animal model according to one embodiment of the present invention.

FIG. 5 is a diagram showing the results of evaluation by scoring four items: piecemeal necrosis, lobular necrosis, histological activity score, and fibrosis, as the observation of liver tissue when the vehicle and Exo-srIκB were administered to the biliary stricture-induced animal model according to one embodiment of the present invention.

FIG. 6 is a diagram showing the measurement of fibrotic area in liver tissue after staining and calculated results thereof as mean and standard deviation when the vehicle and Exo-srIκB were administered to the biliary stricture-induced animal model according to one embodiment of the present invention.

FIG. 7 is a diagram showing the blood analysis on the day of autopsy and calculated results thereof as mean and standard deviation when the vehicle and Exo-SIκB were administered to the biliary stricture-induced animal model according to one embodiment of the present invention.

FIG. 8 is a schematic view of the production of a TAA-induced hepatic fibrosis animal model according to one embodiment of the present invention.

FIG. 9 is a diagram confirming the ICG concentration when the vehicle and Exo-SIκB were administered to the TAA-induced hepatic fibrosis animal model according to one embodiment of the present invention.

FIG. 10 is a diagram showing the results of measuring blood concentrations of aspartate aminotransferase (AST) and alanine aminotransferase (ALT) when the vehicle and Exo-srIκB were administered to the TAA-induced hepatic fibrosis animal model according to one embodiment of the present invention.

FIG. 11 is a diagram showing the results of measuring the change in a-smooth muscle actin (a-SMA), a marker of fibrosis, by Western blotting when the vehicle and Exo-SIκB were administered to the TAA-induced hepatic fibrosis animal model according to one embodiment of the present invention.

FIG. 12 is a diagram confirming hepatic necrosis when the vehicle and Exo-srIκB were administered to the TAA-induced hepatic fibrosis animal model according to one embodiment of the present invention.

FIG. 13 is a schematic view of the production of a DMN-induced liver injury animal model according to one embodiment of the present invention.

FIG. 14 shows increased expression levels of exosome uptake process-related genes in mice and human inflammatory Kupffer cell (KC) according to one embodiment of the present invention. (a-b) Single-cell RNA sequencing (scRNA-seq) of normal mouse liver (GSE132042) was analyzed. Uniform manifold approximation and projection (UMAP) presentation of feature plots for cell type annotation (a) or gene expression levels (b). (c-e) Bulk RNA-seq of mouse primary KC (GSE86397) was analyzed. (c) Kyoto encyclopedia of genes and genomes (KEGG) pathway analysis of upregulated genes in lipopolysaccharide (LPS)-treated KC. (d) Increased gene ontology (GO) biological processes in LPS-treated KC were analyzed. (e) Heatmap showed the relative expression levels of genes related to the indicated pathways. (f, g) scRNA-seq of the human liver (GSE136103) from healthy control (Ctrl, n=5) or patients with alcoholic liver cirrhosis (ALC, n=2) was analyzed. (f) A t-distributed stochastic neighbor embedding (t-SNE) presentation of feature plots showed annotation by group (upper) and expression level of MARCO (lower). (g) Relative expression levels of genes related to indicated pathways were analyzed in MARCO+ KC. For RNA-seq, an adjusted p value was used.

FIG. 15 shows that Exo-srIκB according to one embodiment of the present invention suppresses LPS-induced inflammatory gene expression levels in KC. (a-c) Indicated doses of Dil-labeled Exo-srIκB treated to isolated mouse KC with vehicle (saline, VEH) or LPS (100 ng ml⁻¹) (n=5 per group). (a) Representative pictures of cultured mouse KC. Scale bars, 50 μm. (b,c) The frequencies of Dil+ KC among total KC. (d) Quantitative real-time polymerase chain reaction (qRT-PCR) analyses of isolated mouse KC pretreated with control exosomes (Exo-Naïve) or Exo-srIκB (KC:exosome=1:10,000) for 3 h followed by LPS (100 ng ml⁻¹) for 6 h (n=3 per group). Data are presented as mean±SEM (* p<0.05, * p<0.01, ** p<0.001). Data were analyzed by One-way ANOVA and Tukey's multiple comparisons test.

FIG. 16 is a result confirming that a high dose of Exo-srIκB according to one embodiment of the present invention exhibits an effect of ameliorating alcoholic fatty liver (AFL) and infiltration of inflammatory macrophages. (a-h) Wild-type (WT) male mice were fed EtOH for 10 days and randomly divided into Exo-Naïve or Exo-srIκB group (n=6/group). A single high dose (5.0×10¹⁰ particles per mouse) of exosomes were intravenously injected. After 1 h, mice were given acute EtOH (4 g/kg of 40% EtOH gavage) and sacrificed after 6 h. (a) Schematic diagram of experiments. (b) Representative gross findings and H&E staining of liver tissues. CV means central vein and PT means portal triad. Scale bars, 50 μm. (c) Hepatic triglyceride (TC) levels were measured. (d) Serum levels of alanine aminotransferase (ALT), aspartate aminotransferase (AST), hepatic triglyceride (TG), and total cholesterol (TC). (e) Representative Western blot analyses of hepatic nuclear fraction and densitometry analysis. (f) qRT-PCR analyses of whole liver tissues. (g) The number of mononuclear cell (MNC) per gram of liver tissue. (h) Flow cytometry analyses of liver MNCs with representative panels and bar graphs indicating frequencies or absolute numbers of indicated populations. (i) Result of weight measurement by administration of Exo-srIκB. Data are presented as mean±SEM (* P<0.05, * P<0.01). Data were analyzed by one-way ANOVA and Tukey's multiple comparison test.

FIG. 17 is the result of confirming the mitigation effect of ALI, AFL and ALF in the experimental group injected with Exo-srIκB according to one embodiment of the present invention for 3 consecutive days. (a-f) WT male mice were fed EtOH for 9 days and randomly divided into Exo-Naïve (10⁹ particles/day/mouse), low dose of Exo-srIκB (10⁸ particles/day/mouse) or high dose of Exo-srIκB (10⁹ particles/day/mouse) groups (n=6 per groups). An indicated dose of exosomes was intravenously injected every 24 hours for 3 consecutive days. After 6 h of the last injection, mice were given acute EtOH (4 g/kg of 40% EtOH gavage) and sacrificed after 6 h. (a) Schematic diagram of experiments. (b) Serum levels of ALT and AST were measured. (c) Representative gross findings, H&E, Oil-Red O, and TUNEL staining of liver tissues. Scale bars, 50 μm. In TUNEL staining, apoptotic hepatocytes (HEP) (yellow triangles) and nonparenchymal cell (blue triangles) were indicated. (d) Hepatic TC levels were measured. (e) TUNEL+ apoptosis HEP was counted in 15 random images at ×100 magnification. (f) qRT-PCR analyses of isolated hepatic stellate cell (HSC). Body weight was measured on day 9 (g) or after sacrifice (h). (i) Serum levels of TG and TC were measured. Data are presented as mean±SEM (* P<0.05, * P<0.01, ** P<0.001). Data were analyzed by one-way ANOVA and Tukey's multiple comparison test.

FIG. 18 confirms that the Exo-srIκB according to one embodiment of the present invention is effective in attenuating alcoholic liver inflammation by suppressing the activation of KC in mice. (a-h) Male mice were fed EtOH for 9 days and randomly divided into Exo-Naïve, low-dose Exo-srIκB or high-dose Exo-srIκB groups. An indicated dose of exosomes was intravenously injected every 24 hours for 3 consecutive days. After 6 h of the last injection, mice were given acute EtOH (4 g/kg of 40% EtOH gavage) and sacrificed after 6 h. (a, b) Flow cytometry analyses of F4/80^{hi}CD11b+ KC with C-type lectin-like receptor 2 (CLEC2) and T-cell immunoglobulin and mucin domain containing 4 (TTIM4) with panels (a) and bar graphs (b). (c) Representative C-type lectin domain family 4 member F (CLEC4F) immunostaining of liver tissues. Morphological changes in CLEC4F+ KC were indicated by yellow triangles. (d) Representative Western blot analyses of hepatic nuclear fraction and densitometry analysis. (e) qRT-PCR analyses of isolated KC. (f) Serum IL-6 levels were measured. (g) Representative myeloperoxidase (MPO) immunostaining of liver tissues (upper). MPO+ neutrophils were indicated by blue triangles and counted in 15 random images at ×200 magnification (lower). (h) Flow cytometry analyses of liver MNCs with panels and bar graphs. Data are presented as mean±SEM (* P<0.05, * P<0.01, ** P<0.001). Data were analyzed by one-way ANOVA and Tukey's multiple comparison test. Scale bars, 50 μm.

FIG. 19 confirms that the Exo-srIκB according to one embodiment of the present invention shows the effect of reducing infiltration of immune cell into the liver. (a) qRT-PCR analyses of isolated HEP. (b) The number of MNC cell per gram of liver tissues was analyzed. (c-e) Flow cytometry analyses. Data are presented as mean±SEM (*P<0.05, *P<0.01, **P<0.001). Data were analyzed by one-way ANOVA and Tukey's multiple comparison test. Scale bars, 50 μm.

### BEST MODEL

Hereinafter, the present invention will be described in detail. Meanwhile, each description and embodiment disclosed herein can be applied to other descriptions and embodiments, respectively. That is, all combinations of various elements disclosed herein fall within the scope of the present invention. Further, the scope of the present invention is not limited by the specific description described below.

Additionally, those of ordinary skill in the art may be able to recognize or confirm, using only conventional experimentation, many equivalents to the particular aspects of the invention described herein. Furthermore, it is also intended that these equivalents be included in the present invention.

One aspect for implementing the present invention provides a pharmaceutical composition for preventing or treating liver disease, including an exosome. Specifically, the exosome refers to an exosome containing super-repressor-IκB (srIκB). The super-repressor-IκB (srIκB) is an active ingredient providing a preventive or therapeutic effect for liver disease, which is contained in the exosome of the present invention, and may be used interchangeably with cargo protein.

The exosome may contain super-repressor-IκB (SrIκB).

As used herein the term “super-repressor-IκB (SrIκB)” is a protein that is not phosphorylated by IκB kinase (IKK) and is not degraded by proteasome, and is in the S32A, S36A mutant form of IκB protein (IκBα). In the present invention, SrIκB includes IκB protein or fragments thereof. The super-repressor-IκB (SrIκB) may be an amino acid sequence of SEQ ID NO: 1 or 2, or an amino acid sequence having at least 95% sequence homology thereto, or a fragment thereof, but is not limited thereto.

Hereinafter, the srIκB may be expressed as a cargo protein.

As used herein, the term “exosome” refers to a vehicle capable of loading cargo proteins, and may be loaded with cargo proteins by various methods known in the art.

Examples of known methods for loading cargo proteins include: a method of overexpressing cargo proteins in an exosome-producing cell, thereby loading into the exosome; a method of overexpressing a fusion protein in an exosome-producing cell using a vector in which an exosome-specific marker and a cargo protein are fused to increase loading efficiency, thereby loading into the exosome; or a method of loading cargo proteins into the exosome through photodynamically reversible protein-protein interactions, etc., but are not limited thereto.

Specifically, as methods for producing exosomes containing the super-repressor-IκB (SrIκB) protein of the present invention, the present invention may be incorporated with the full disclosure of U.S. patent Ser. No. 10/702,581 and Korean Patent No. 10-1733971 by reference to provide compositions and methods for preparing the exosome disclosed herein. In the present invention, the “exosome containing the super-repressor” can be used interchangeably with “Exo-srIκB”.

The exosome may have a diameter of about 50 nm to about 200 nm, specifically about 50 nm to about 150 nm, but is not limited thereto.

As used herein, the “liver disease” refers to a disease that occurs in the liver, specifically, it may include liver fibrosis, liver cirrhosis, fatty liver, alcoholic liver disease, cholestasis, or a combination thereof, but is not limited thereto as long as abnormalities occur in liver tissue and function.

As used herein, the “liver fibrosis” refers to the result of the wound healing process for repeated liver damage. It is known that liver fibrosis is reversible unlike liver cirrhosis, is composed of thin fibrils, and does not have nodule formation, and the liver may be recovered to the normal condition when the cause of liver damage is eliminated. However, when this liver fibrosis process continues repeatedly, liver fibrosis progresses to liver cirrhosis.

As used herein, the “liver cirrhosis” is a chronic disease that occurs with repeated increasing of the regeneration of liver cell and fibrous tissue, it is pathologically accompanied by necrosis, and fibrosis, and ultimately progresses to cirrhosis complications such as liver failure and diseases such as hepatocellular carcinoma, leading to death. In particular, since liver cirrhosis is discovered only after considerable progress due to the absence of awareness of one's own symptoms in the earty stages of the disease, it is very difficult to be treated.

As used herein, the “fatty liver” refers to a disease in which fat is accumulated in liver cell, and specifically, it may include alcoholic fatty liver caused by excessive drinking and non-alcoholic fatty liver caused by obesity, diabetes, hyperlipidemia, or metabolic syndrome.

As used herein, the “alcoholic liver disease” refers to a liver disease that occurs due to excessive drinking, and specifically, it may be a variety of diseases such as alcoholic fatty liver, alcoholic steatohepatitis, alcoholic cirrhosis, and alcoholic liver fibrosis, but is not limited thereto.

Specifically, the “alcoholic liver disease” refers to several groups of liver diseases caused by chronic alcohol consumption. As the first stage of alcoholic liver disease, alcoholic fatty liver, in which fat is accumulated in liver cell due to continuous alcohol consumption, occurs. As the disease progresses, severe fat accumulation, hepatic necrosis, and acute inflammatory reaction appear, and these stages are referred to as alcoholic hepatitis. Thereafter, as the final step, collagen in liver tissue accumulates and may progress to alcoholic cirrhosis which is accompanied by liver fibrosis. Therefore, such alcoholic liver disease rarely progresses sequentially or appears as a single disease, and in most cases, the progressive stages overlap and appear in the form of a complex disease group.

As used herein, the “cholestasis” refers to a disease in which bile is stalled in the liver due to autoimmune disease or biliary obstruction. Cholestatic diseases can be largely divided into extrahepatic cholestasis (external compression of the biliary tract, internal obstruction of the biliary tract, biliary stenosis, Caroli's disease, etc.) and intrahepatic cholestasis. Cholestasis can be used interchangeably with cholestasis liver disease. The cholestasis liver disease includes, in order of decreasing frequency, primary biliary cholangitis (PBC), primary sclerosing cholangitis (PSC), progressive familial intrahepatic cholestasis (PFIC), and Alagille syndrome (AS).

The composition of the present invention may exhibit (a) reduction in the relative weight of the liver; (b) reduction of jaundice; (c) decrease in liver abscess levels; (d) reduction of liver fibrosis; (e) decrease in T-BIL levels; (f) increase in HDL levels; (g) decrease in quantitative changes in a-SMA in hepatic stellate cells; (h) decrease in serum ALT/AST levels; and/or (i) reduction of alcoholic fatty liver, thereby preventing or treating liver disease, but is not limited thereto.

The exosome of the present invention or a pharmaceutical composition including the exosome may further include a pharmaceutically acceptable excipient. The pharmaceutically acceptable excipient may include, for oral administration, a binder, a lubricant, a disintegrant, a solubilizing agent, a dispersant, a stabilizing agent, a suspending agent, a coloring agent, a fragrance, etc.; for injections, a buffering agent, a preservative, an analgesic, a solubilizing agent, an isotonic agent, a stabilizing agent, etc., which may be combined to be used; and for topical administrations, a base, an excipient, a lubricant, a preservative, etc., although it is not particularly limited thereto.

The formulation type of the composition of the present invention may be prepared variously by being combined with a pharmaceutically acceptable excipient described above. For example, the composition of the present invention may have any one formulation type selected from the group consisting of tablets, pills, powders, granules, capsules, suspensions, liquid medicine for internal use, emulsions, syrups, sterile aqueous solutions, non-aqueous solvents, lyophilized formulations, and suppositories. The composition of the present invention may be a physiologically acceptable aqueous solution or suspension of exosomes. In the case of an injection, the composition may be formulated into unit-dose ampoules or multi-dose forms.

Additionally, the composition may be formulated into a preparation of a unit dosage form suitable for the administration into a patient's body according to the conventional method in the pharmaceutical field so as to be administered by an oral or parenteral route (including skin, intravenous, intramuscular, intraarterial, intramedullary, intrathecal, intraventricular, pulmonary, transdermal, subcutaneous, intraperitoneal, intranasal, intragastrical, topical, sublingual, vaginal, or rectal route) using the conventional administration method in the art, but the administration routes are not limited thereto.

The administration dose and frequency of the pharmaceutical composition of the present invention are determined by the type of drugs, which are active ingredients, together with various factors, such as the disease to be treated, administration route, patients age, sex, and body weight, severity of the disease, etc. Specifically, the composition of the present invention may contain the exosomes in a pharmaceutically effective amount, but is not limited thereto.

Containing the exosomes in a pharmaceutically effective amount refers to a level at which the desired pharmacological activity (e.g., prevention, improvement, or treatment of liver disease) can be obtained by the exosomes of the present invention, and may refer to a pharmaceutically acceptable level, which is a level at which toxicities or adverse effects do not occur or occur at an insignificant level in the subject to be administered, but the level is not limited thereto. The pharmaceutically effective amount may be determined by comprehensively considering the number of administrations, patient, formulations, etc.

Although not particularly limited thereto, the pharmaceutical composition of the present invention may contain the above ingredients (active ingredients) in an amount of 0.01% (W/V) to 99% (W/V).

The total effective dose of the composition of the present invention may be administered to a patient in a single dose. In the pharmaceutical composition of the present invention, the content of the active ingredient(s) may vary depending on the severity of the disease. Specifically, the total daily dose of the exosome of the present invention may be about 1×10⁶ to 1×10¹⁵ pn per 1 kg of the body weight of a patient. However, the effective dose of the exosome is determined considering various factors including patients age, body weight, health conditions, sex, disease severity, diet, and excretion rate, as well as administration route and treatment frequency of the pharmaceutical composition. In this respect, those skilled in the art may easily determine the effective dose suitable for a particular use of the composition of the present invention. The pharmaceutical composition according to the present invention is not particularly limited to the formulation type, administration route and mode, as long as it shows the effects of the present invention.

Another aspect for implementing the present invention provides a method for preventing or treating liver disease, including an exosome containing super-repressor-IκB (srIκB) protein.

### MODE FOR INVENTION

Hereinafter, the Preparation Examples are only for describing the present invention in more detail, and the scope of the present invention is not limited by these Preparation Examples.

### Preparation Example 1. Preparation of Exosome (Exo-srIκB) Containing Target Protein

An exosome containing super-repressor-IκB (srIκB) as a target protein was prepared by the method described in Korean Patent No. 10-1877010.

### Preparation Example 2. Preparation of Control Exosome (Exo-naïve

Expi293F cells (Thermofisher) were incubated for 4 days under light irradiation conditions and then the culture medium was harvested and purified to obtain Exo-naïve in the same manner as in Preparation Example 1.

Hereinafter, the present invention will be described in more detail with reference to the following Examples. However, these Examples are only for describing the present invention in more detail, and the scope of the invention is not limited by these Examples

### Example 1. Efficacy Evaluation of srIκB-Loaded Exosomes (Exo-srIκB) in Minipig Model of Biliary Stricture-Induced Liver Cirrhosis

### 1-1. Construction of Biliary Stricture-Induced Animal Model

In order to confirm whether the exosome containing the super-repressor (srIκB) of the present invention can prevent or treat liver disease, an animal model in which liver disease was induced by various causes was constructed for confirmation.

First, an animal model in which biliary stricture was induced was constructed to confirm the effectiveness of the exosome (Exo-SrIκB) containing the super-repressor (SrIκB).

Specifically, Endobiliary Radiofrequency Ablation (EB-RFA) guided Endoscopic Retrograde Cholangiogram (ERC) was performed on the minipig using an RFA catheter (ELRA RF catheter; STARmed) with a temperature sensing system to construct a biliary stricture-induced minipig model. The area in which the inserted RFA electrode and the wall of the bile duct meet was designated as EB-RFA, and radiofrequency energy was performed at 80° C., 7 W for 90 seconds using the device (Table 1 below).

TABLE 1

| | | Control/Test | | |
|---|---|---|---|---|
| Group | RFA | article | Treatment (i.v.) | |
| 1 | 80° C., 7 W, | Vehicle | Buffer | 1 mL/kg |
| | 90 sec | control | | |
| 2 | | Test article | Exo-srlKB | 1.6e+10 |
| | | | | pn/kg/1 mL |
| 3 | None | Normal control | None | None |

At 3 weeks after RFA induction, a stent (Nexent biliary stent, total length: 4 cm) was inserted into the EB-RFA to open the obstructed biliary tract, and the animals were necropsied at 5 weeks after RFA induction.

After RFA induction, the test substance (vehicle-buffer, Exo-srIκB) was intravenously administered (intravenous, i.v.) for 1 hour using an infusion pump for a total of 4 times at 1, 2, 3, and 4 weeks, and the dose for each individual was converted based on the body weight immediately before administration, which was converted into a corresponding concentration of 1.6e+10 pn/kg/1 mL.

Based on RFA, blood was collected 6 times before administration (vehicle-buffer, Exo-srIκB) before induction (0 hour), and at 1, 2, 3, 4, and 5 weeks.

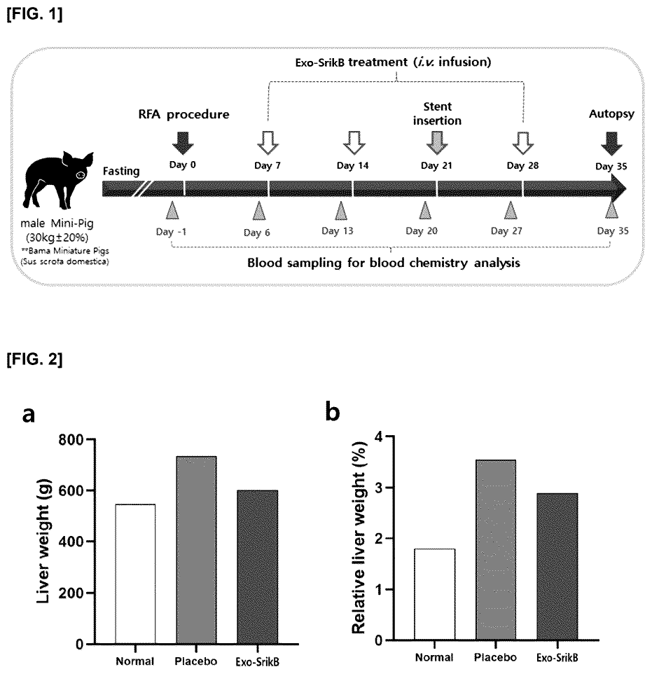

The detailed animal model production method is shown in FIG. 1.

1-2. Confirmation of Relative Weight of Liver and Liver Tissue Findings after Autopsy and Histopathology

After the test was completed, the animals were anesthetized, and blood was taken from the vein and exsanguinated, and then the liver tissue was excised for each individual. The weight of the extracted liver tissue was measured and photographed (Appendix 9.8). Then, the excised liver tissue was fixed in 10% neutral buffered formalin. The fixed tissue was sliced to a certain thickness, and subjected to paraffin-embedding through a general tissue treatment process to prepare tissue sections of 4-5 μm, followed by H&E (Hematoxylin & Eosin staining) staining, which is a staining method.

Next, the relative weight (%) of the liver was measured and calculated as mean, and the results are shown in FIG. 2.

As a result, as can be seen in FIG. 2, the relative weight of G1 (vehicle-control group) was 3.5, G2 (Exo-srIκB-administered group) was 2.9, and G3 (normal-control group) was 1.8, confirming that the relative weight of G1 (vehicle-control group) was increased by 2 times as compared to G3 (normal-control group) and that the relative weight of G2 (Exo-srIκB-administered group) was decreased by 17% compared to G1 (vehicle control group) (FIG. 2b).

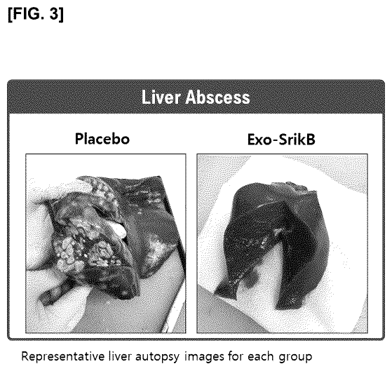

Additionally, as a result of confirming liver abscess as a clinical symptom, as can be seen in FIG. 3, liver abscess was observed in the vehicle control group, but was not found in the Exo-srIκB-administrated group of the present invention.

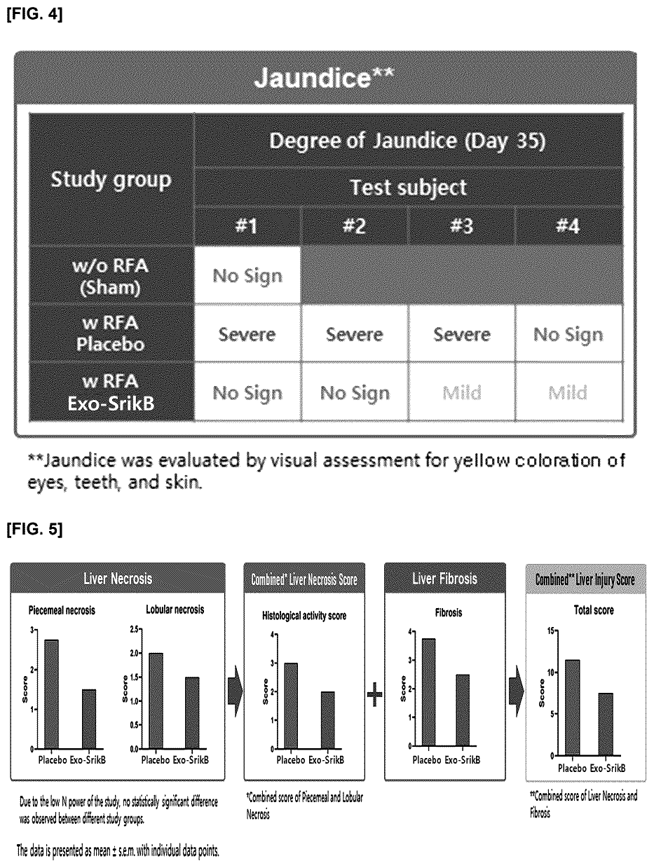

In addition, as a result of confirming the symptoms of jaundice as a clinical symptom, as can be seen in FIG. 4, the symptoms of jaundice were observed in the gums and the sclera of the vehicle-control group, but were not found in the Exo-srIκB-administered group of the present invention.

As a result of observing subcutaneous sections after laparotomy on the day of autopsy, jaundice, which was not found during the observation period, was additionally confirmed in the vehicle-control group and the Exo-srIκB-administered group. Further, it was confirmed that jaundice was slightly observed in the Exo-srIκB-administered group than in the vehicle-control group.

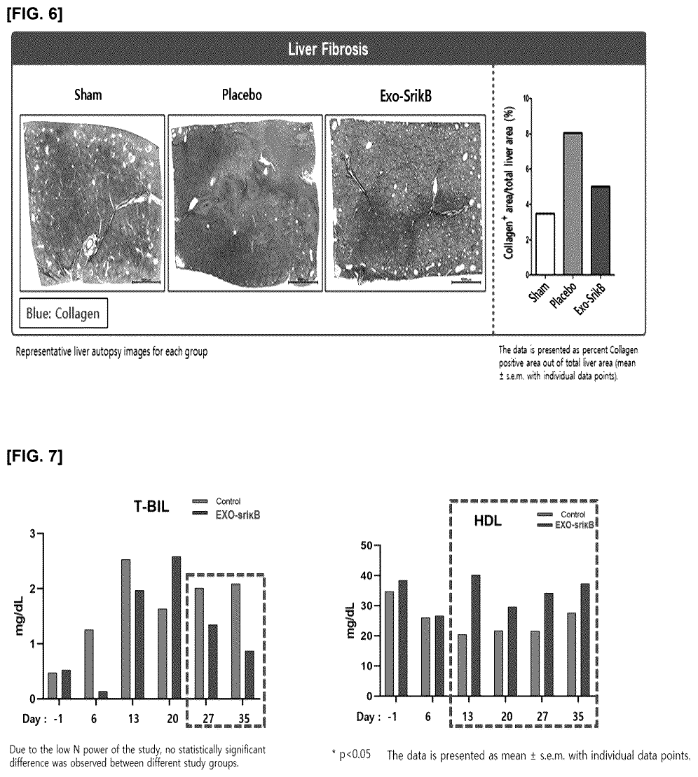

Next, as the findings of observing the liver tissue, the liver tissue was evaluated by scoring on four items: piecemeal necrosis, lobular necrosis, histological activity score, and fibrosis, and scoring them, and the results are shown in FIG. 5.

As can be seen in FIG. 5, as a result of calculating the scores observed in liver tissue as mean, in the case of G1 (vehicle-control group), the piecemeal necrosis score was 2.8, the lobular necrosis score was 2.0, the histological activity score was 3.0, and the fibrosis score was 3.8, adding to the total score of 11.5.

In contrast, in the case of G2 (Exo-srIκB-administered group), the piecemeal necrosis score was 1.5, the lobular necrosis score was 1.5, the histological activity score was 2.0, and the fibrosis score was 2.5, adding to the total score of 7.5.

Based on the results, it was confirmed that G2 of the present invention showed decreased piecemeal necrosis score by 46%, decreased lobular necrosis score by 25%, decreased histological activity score by 33%, decreased fibrosis score by 34% as compared to G1 (vehicle-control group), and the total score was decreased by 35%.

### 1-3. Confirmation of Collagen Fiber Distribution

In order to confirm the distribution of collagen fibers, MT (Masson's Trichrome) staining, which stains only collagen fibers, was performed. The tissue slide for each individual was photographed as a whole, and the stained area was measured relative to the total area, and the average value was calculated.

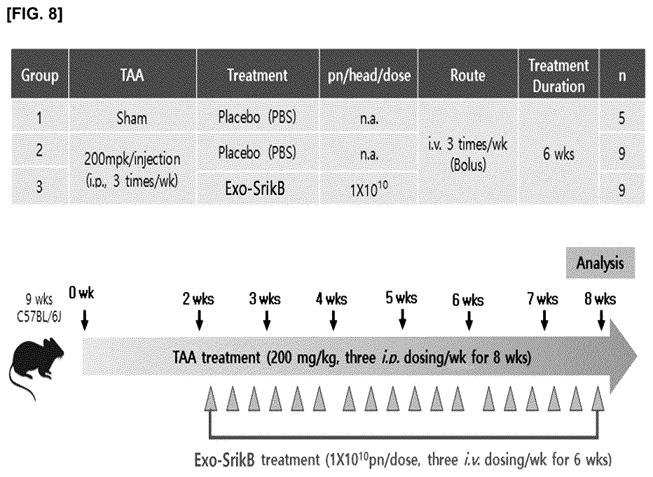

The fibrotic sections in the liver tissue were stained and measured (%), which were calculated as mean and standard deviation, and the results are shown in FIG. 6.

As a result, as can be seen in FIG. 6, the collagen area of G1 (vehicle-control group) was 8.1, the collagen area of G2 (Exo-srIκB-administered group) was 5.0, and the collagen area of G3 (normal control group-Sham) was 3.5, confirming that the collagen area of G2 (Exo-srIκB-administered group) of the present invention was decreased by 38.3% compared to that of G1 (vehicle-control group).

### 1-4. Confirmation of Blood Biochemistry Test

The blood obtained in Example 1-1. was centrifuged at 3000 rpm, 4° C. for 10 minutes to obtain the serum in the supernatant, and then T-Total bilirubin (BIL) and high-density lipoprotein cholesterol (HDL-C) were measured for a total of 11 items using an automated blood biochemistry analyzer.

Blood biochemistry tests were conducted before the RFA procedure, before administration of test substance, and on the day of autopsy. The blood was analyzed on the day of autopsy and calculated as mean and standard deviation, and the results are shown in FIG. 7.

As can be seen in FIG. 7, in G1 (vehicle-control group), T-BIL was found to be 2.09 mg/dL and HDL-C was found to be 27.7 mg/dL, and in G2 (Exo-srIκB-administered group), T-BIL was found to be 0.87 mg/dL and HDL-C was found to be 37.4 mg/dL.

In most measurement items, there was no difference between G1 (vehicle-control group) and G2 (Exo-srIκB-administered group) of the present invention, but in G2 (Exo-sIκB-administered group), the T-BIL concentration was decreased by 2.4 times and the HDL level was increased by 1.4 times as compared to G1 (vehicle-control group).

In summary, in the biliary stricture-induced liver cirrhosis animal model, it was confirmed that the composition including the exosome (Exo-srIκB) containing the super-repressor (srIκB) of the present invention had an effect of reducing the relative weight (%) of the liver and reducing liver abscess compared to the vehicle-control group, and showed effective reduction of liver fibrosis in MT staining that confirms liver tissue fibrosis. As a result of the blood biochemistry test, the Exo-sIκB significantly reduced the T-BIL, while exhibiting the effect of increasing HDL. Based on the above results, it can be confirmed that the Exo-srIκB of the present invention effectively treats fatty liver, cholestasis, liver fibrosis and liver cirrhosis, and further exhibits excellent effects of protecting the liver and improving the function of the liver.

### Example 2. Effectiveness Evaluation of Exo-srIκB Using TAA-Induced Liver Fibrosis Animal Model

### 2-1. Construction of Animal Model

Next, an animal model of liver fibrosis induced by TAA (Thioacetamide (172502-500G, SIGMA)) was prepared and the effectiveness of Exo-srIκB of the present invention was evaluated.

Specifically, TAA was intraperitoneally administered to mice (C57BL/6, 9-week-old, male) at a dose of 200 mg/kg three times a week for 8 weeks to prepare an animal model of liver fibrosis.

Since it confirmed that the increase in blood biochemistry biomarker and the increase in histopathological fibrosis started 2 weeks after TAA 200 mg/kg administration as a preliminary experiment, the timing of Exo-srIκB administration was determined to be 2 weeks after TAA administration. The administration of Exo-srIκB was carried out by intravenous bolus administration 3 times a week for 6 weeks. Since the administration time of Exo-srIκB was the same as TAA, TAA was administered in the morning and Exo-sIκB was administered in the afternoon. Thereafter, the body weight of the animals was measured for 8 weeks, and the dose was administered according to the body weight, and Exo-srIκB was administered at a dose of 4E+11 pn/kg.

When 200 mg/kg of TAA was administered, the mortality rate of animals reached 20% to 30% within 2 weeks, and thus, groups were formed around 1.5 weeks after TAA administration, which was before Exo-srIκB administration. The groups were composed of a mock control group (Sham), a negative control group administered only with 200 mg/kg of TAA, and a group administered with Exo-srIκB. Nine animals were selected for each group. Among them, 4 animals in each group were used for the indocyanine green (ICG) test.

At week 8 of termination of the experiment, an autopsy was performed, whole blood was collected, and serum was isolated. Then, the liver of the mice was excised to separate proteins, and some were fixed for histopathological experiments. Four animals per group for the ICG test were tested the next day, and 5 mg/kg of ICG was administered intravenously, and blood was collected after 15 minutes to measure the concentration of ICG present in the blood.

The construction of the animal model is shown in FIG. 8.

### 2-2. Analysis Result of ICG Test

More than 90% of ICG is ingested by hepatocytes and excreted only by the liver, and the retention rate after ICG administration is used as an index to evaluate liver function.

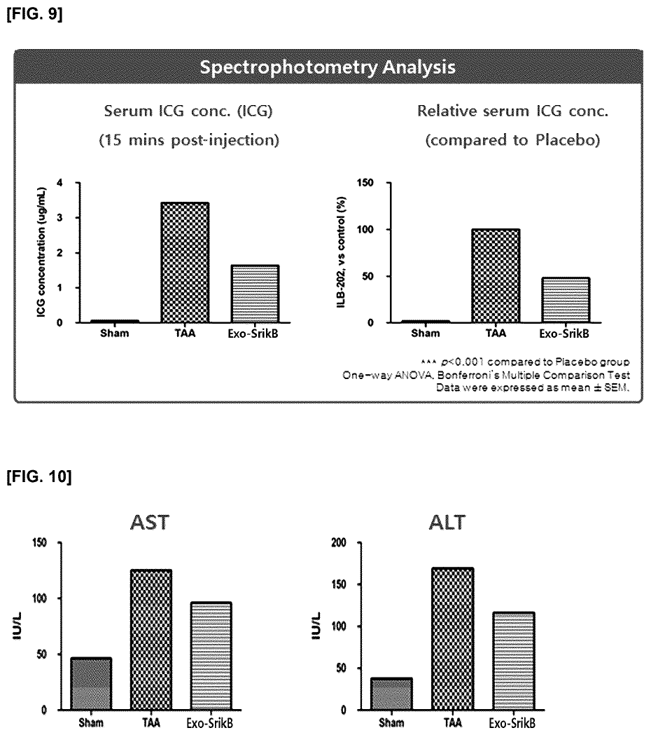

Specifically, after administering 5 mg/kg of ICG to the animals, blood was collected 15 minutes later to evaluate the ICG concentration, and the results are shown in FIG. 9.

As a result, as can be seen in FIG. 9, it was confirmed that the concentration of ICG in the blood of the negative control group was increased compared to that of the mock control group. In contrast, in the Exo-srIκB-administered group of the present invention, it was confirmed that the ICG concentration was significantly reduced by about 52.9% compared to the negative control group.

### 2-3. Analysis Result of Blood Biochemical Biomarker

The blood concentrations of aspartate aminotransferase (AST) and alanine aminotransferase (ALT) as markers of liver damage were measured, and the results are shown in FIG. 9.

For reference, AST and ALT are aminotransferases present in the liver that are involved in gluconeogenesis and are released into the blood when the liver is damaged, and thus are enzymes for diagnosing liver diseases.

As a result, as can be seen in FIG. 10, it was confirmed that AST and ALT were increased in the negative control group compared to the mock control group, whereas they were suppressed in the Exo-srIκB-administered group of the present invention.

### 2-4. Analysis Results of Fibrosis Marker

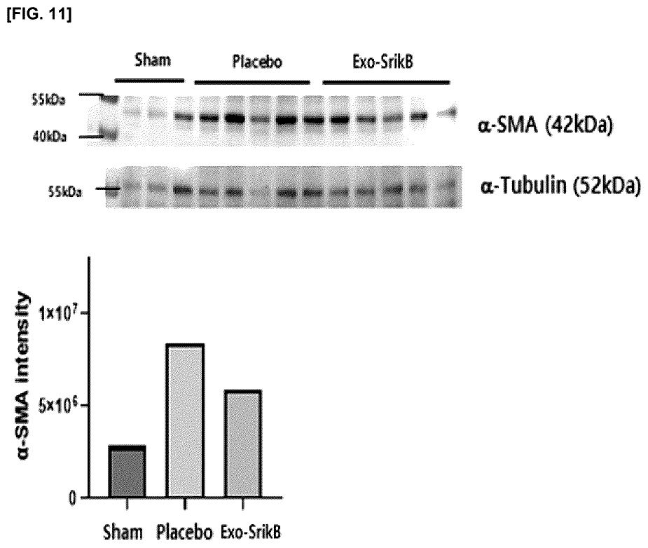

Proteins were isolated from the mouse liver, and the changes in a-SMA (a-smooth muscle actin), a marker of fibrosis, were measured by Western blotting, and the amount of protein loaded in the experiment was normalized and analyzed as the amount of a-tubulin. The results are shown in FIG. 11.

As can be seen in FIG. 11, it was confirmed that the amount of a-SMA in the liver tissue of the negative control group increased compared to the mock control group, whereas the amount of a-SMA in the Exo-srIκB-administered group of the present invention decreased.

### 2-5. Analysis Results of Hepatic Necrosis

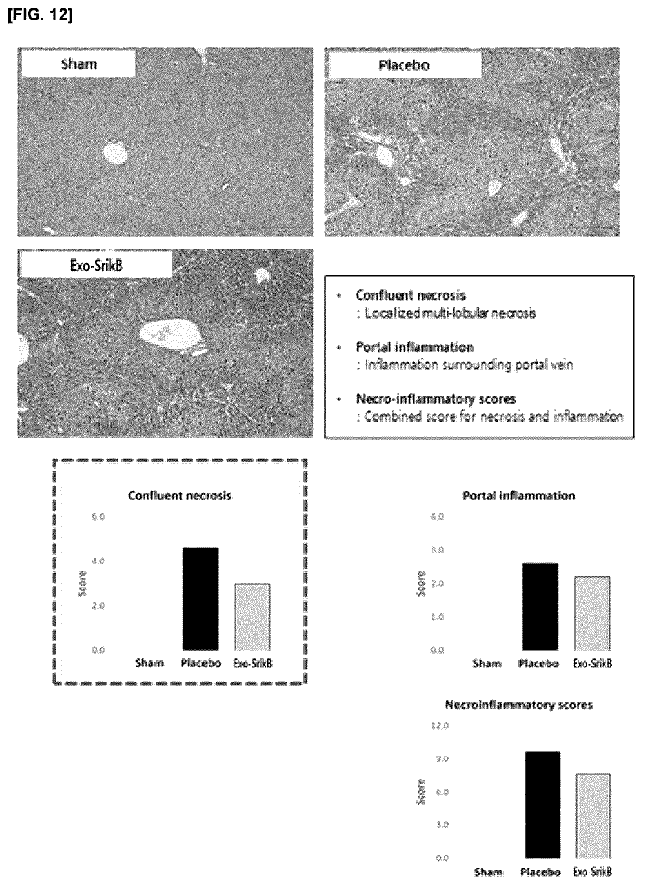

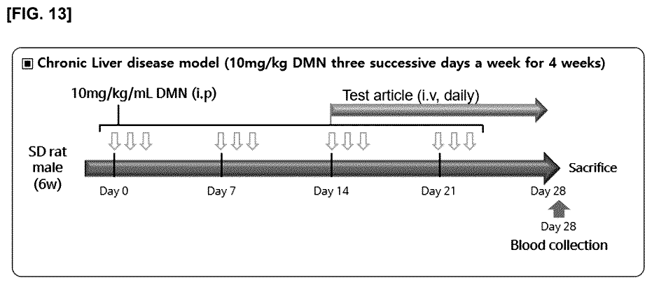

The fixed liver tissue was subjected to Hematoxylin-Eosin staining (H&E staining) to evaluate the degree of hepatic necrosis, which was scored by a histopathology expert. The results are shown in FIG. 12.

As can be seen in FIG. 12, it was confirmed that hepatic necrosis in the negative control group was significantly increased compared to the mock control group, but was reduced by the Exo-srIκB-administered group of the present invention.

In summary, in the liver fibrosis animal model prepared by intraperitoneal administration of TAA, which causes liver damage, to the mice, the blood concentrations of AST and ALT associated with liver damage were significantly reduced in the group administered with Exo-srIκB of the present invention. In addition, as a result of intravenous administration of ICG, which is excreted only by the liver, and measurement of blood concentration, it was confirmed that the concentration of ICG in the blood was significantly reduced in the Exo-srIκB-administered group of the present invention compared to the negative control group.

Further, as a result of analyzing the amount of a-SMA, it was confirmed that a-SMA was increased in the negative control group compared to the mock control group, but decreased in the group administered with Exo-srIκB. As a result of scoring the quantitative changes of necrotic cells, it was confirmed that the Exo-srIκB-administered group showed a reduction effect compared to the negative control group.

From the above results, Exo-srIκB can be effectively used for the treatment of liver fibrosis, liver cirrhosis, and fatty liver by inhibiting liver cell necrosis and liver fibrosis.

### Example 3. Evaluation of Exo-srIκB Effectiveness Against Alcoholic Liver Disease (ALD)

### 3-1. Construction of Animal Model

In order to confirm the therapeutic efficacy of Exo-SrIκB of the present invention in an acute/chronic alcoholic liver injury mouse model (Ethanol-induced Steatohepatitis, NIAAA model), an animal model was prepared as follows.

Specifically, 8- to 10-week-old C57BL/6JWT male mice approved by approved by the Institutional Animal Care and Use Committee of the Korea Advanced Institute of Science and Technology (KAIST) were used. The mice were fed 4.5% EtOH and subjected to liquid diet feeding for 10 days.

For a 3-day consecutive infusion experiment, the mice were randomly divided into groups of Exo-Naïve (10⁹ particles/day/mouse), low-dose of Exo-sdIκB (10⁶ particle/day/mouse), or high-dose Exo-srIκB (10⁹ particle/day/mouse). The indicated doses of exosomes were intravenously delivered for 3 consecutive days with 24-hour intervals. Mice were given acute EtOH (4 g/kg of 40% EtOH gavage) after 6 hours of last exosome injection and sacrificed after 6 hours.

Hepatocyte (HEP), hepatic stellate cell (HSC), Kupffer cell (KC), and liver mononuclear cell (MNC) were isolated from C57BL/6J WT male mice.

### 3-2. Confirmation of Major Target Cell Types of Exo-srIκB

The transcriptional profiles of diverse hepatic cells were examined to investigate the major target cell types for Exo-srIκB using scRNA-seq analysis.

Specifically, single-cell RNA sequencing (scRNA-seq) analysis of normal mouse liver tissues can be explored on the Tabula Muis Senis website (https://tabula-muris-senis.ds.czbiohub.org/) or found in NCBI Gene Expression Omnibus under accession number GSE132042.

Kyoto encyclopedia of genes and genomes (KEGG) pathway and gene ontology analyses were performed with Database for Annotation, Visualization and Integrated Discovery (DAVID) (https://david.ncifcrf.gov/). The scRNA-seq analysis of human liver specimens is publicly available in NCBI Gene Expression Omnibus under accession number GSE136.

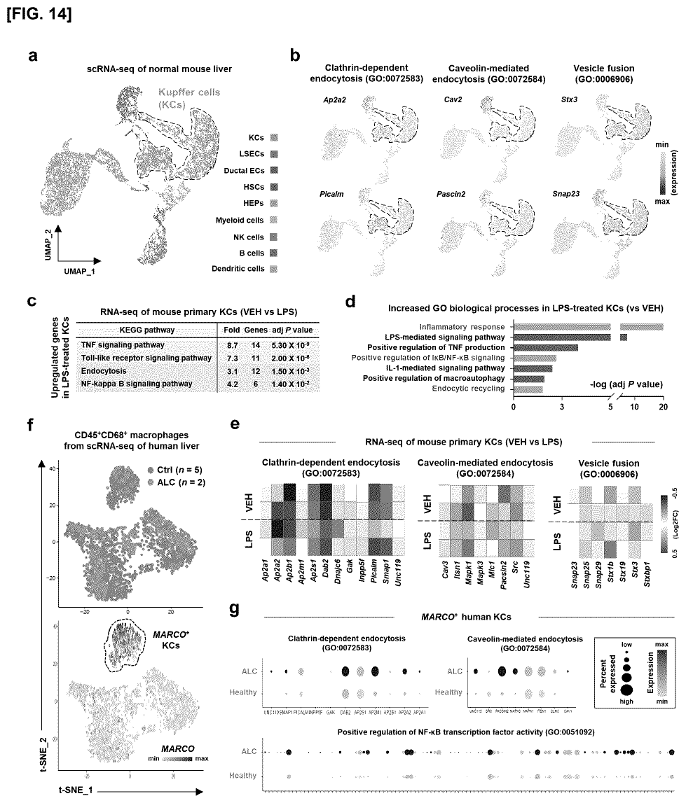

When analyzing the scRNA-seq of normal mouse liver, a total of nine clusters were confirmed as KC, liver sinusoidal endothelial cell (LSEC), ductal epithelial cell, hepatic stellate cell (HSC), hepatocyte (HEP), myeloid cell, natural killer (NK) cell, B cell, and dendritic cell (FIG. 14a).

Additionally, it was confirmed that the expression levels of genes related to exosome uptake processes, such as clathrin-dependent endocytosis (Ap2a2 and Picalm), caveolin-mediated endocytosis (Cav2 and Pascin2), and vesicle fusion (Stx3 and Snap23), were enriched in LSEC or KC (FIG. 14b). Due to the anatomical location of KC over the LSECs, it might be plausible that KC might primarily take up Exo-srIκB after intravenous injection.

### 3-3. Confirmation of Increase in Expression Level of Genes Related to Exosome Uptake Process

Next, in order to confirm whether inflammatory activation of KC could affect the expression levels of genes related to exosome uptake processes, bulk RNA-seq analysis of KC was performed. The bulk RNA-seq of a vehicle or LPS-treated mouse primary KC is publicly available in NCBI Gene Expression Omnibus under Accession Number GSE86397.

As a result, the expression levels of genes related to clathrin-dependent or caveolin-mediated endocytosis pathways were elevated. In addition, the expression levels of genes in the inflammatory responses and NF-kB signaling pathways in lipopolysaccharide (LPS)-treated KC were elevated compared to those of vehicle-treated KC (FIG. 14c-e). Further, expression levels of genes related to clathrin-dependent endocytosis, caveolin-mediated endocytosis, and positive regulation of NF-κB transcription factor activity were upregulated in scRNA-seq analysis of MARCO+ human KC isolated from patients with liver cirrhosis compared to those of controls (FIG. 14f and FIG. 14g).

Based on the results, it can be found that Exo-srIκB can be efficiently delivered to activated KC and inhibit NF-κB-mediated inflammation in ALD.

### 3-4. Confirmation of Inhibition of Inflammatory Gene Expression in LPS-Treated Mice In Vitro

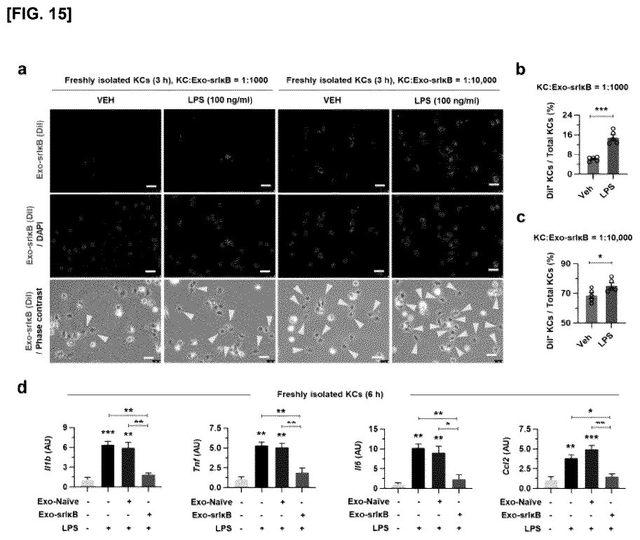

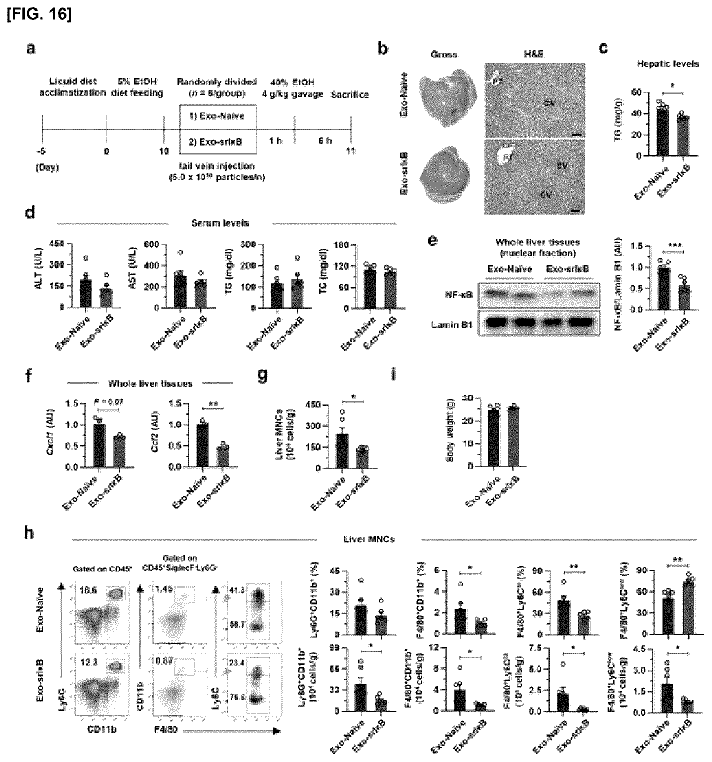

The efficient delivery of Exo-srIκB in KC and its inhibitory effect on LPS-induced inflammatory gene expression levels in vitro were confirmed. The delivery of Exo-srIκB in KC was confirmed by Dil labeling. Specifically, isolated and purified Exo-srIκB (1.0×10¹² particles) was incubated with 10 μL of Dil at 37t for 30 min while being protected from light. After that, 1× filtered phosphate-buffered saline was added to up to 4 mL and moved to Amicon® Ultra-4 centrifugal filter unit (Merck, Rahway, NJ, USA). Solutions were eluted by centrifugation at 3200×g, 4° C., for 15 min until the residual volume reached approximately 200 μL. First, to investigate the delivery of EXO-SrIκB by KC, the Dil-labeled Exo-srI_B was treated to mouse primary KC in time (1 or 3 hours)- and dose (KC: Exo-srIκB=1:1000 or 1:10,000)-dependent manners. Although the Exo-srIκB-laden KC could not be detected after 1 hour, it was confirmed that EXO-srIκB was delivered to about 8% of KC at low dose (KC: Exo-srIκB=1:1000) and 70% at high dose (KC: Exo-srIκB=1:10,000) after 3 hours of treatment (FIG. 15a-c).

To explore the suppressive role of EXO-srIκB on LPS-induced inflammatory activation in KC, Exo-Naïve or Exo-srIκB was pretreated to mouse KC followed by LPS, and qRT-PCR was performed.

The qRT-PCR analyses revealed that the treatment of Exo-srIκB normalized the increased expression levels of inflammatory genes (Il1b, Tnf, Il6, and Ccl2) by LPS (FIG. 15d). In addition, it was confirmed that the LPS treatment significantly enhanced the uptake of EXO-srIκB by KC (FIG. 15a-c).

Based on the results, it can be found that Exo-srIκB is effectively taken up by KC cell activated by inflammation.

### 3-5. Confirmation of Therapeutic Effect of Alcoholic Fatty Liver (AFL) and Alcoholic Liver Injury (ALI) by Administration of Exo-srIκB

Prompted by the above findings from the previous experiments, the therapeutic potential of Exo-srIκB in an acute-on-chronic ALI experimental model was confirmed. After 10 days of EtOH feeding, the mice were randomly divided into 2 groups and intravenously injected Exo-Naïve or Exo-srIκB (FIG. 16a). For histological analyses, similar regions of the left and medial lobes of mouse liver were used. Liver tissues were fixed with 10% neutral buffer formalin (Sigma-Aldrich, St. Louis, MO, USA) overnight at room temperature. After deparaffinization and rehydration, the paraffin-embedded tissues were sliced at 4 μm thickness and subjected to Hematoxylin & Eosin (H&E) staining.

As a result, a significant decrease in alcoholic fatty liver (AFL) was confirmed in Exo-srIκB-treated mice compared to that of Exo-Naïve-treated mice as confirmed by hepatic TG measurement (FIG. 16b and FIG. 16c).

Additionally, for biochemical analysis, haptic triglyceride (TG) was measured. Specifically, hepatic lipids were extracted from about 20 to 30 mg of frozen liver tissues using chloroform/methanol (2:1 ratio) solution. Then, lyophilized hepatic lipids were suspended again to 5% bovine serum albumin (BSA)-saline. The VetTest Chemistry analyzer (IDEXX Laboratories, Westbrook, ME, USA) was used to measure the hepatic TG levels and serum levels of alanine aminotransferase (ALT), aspartate aminotransferase (AST), TG, and total cholesterol (TC).

As a result, the serum levels of ALT and AST were reduced by Exo-srIκB, and there were no differences in serum TG and TC levels and body weight between the two groups (FIG. 16d and FIG. 16i).

Next, in order to confirm the hepatic delivery of Exo-srIκB, the NF-kB levels in the nuclear fraction of whole liver tissues was measured by Western blotting. Nuclear protein levels were normalized to the expression of lamin B1 for each sample. Densitometry analysis was performed with ImageJ (National Institute of Health, Bethesda, MD, USA).

As a result, significantly decreased nuclear translocation of NF-kB was detected in Exo-srIκB-treated mice (FIG. 16e). Additionally, the hepatic expression levels of CXCL1 and CCL2 were suppressed, resulting in a lower number of MNCs per gram of liver tissue in Exo-srIκB-treated mice compared to that of the control mice (FIG. 16f and FIG. 16g).

Further, flow cytometry analyses were performed to confirm whether hepatic frequencies of infiltrating macrophages were decreased. As a result, a decrease in hepatic frequencies of F4/80⁺CD11b⁺ infiltrating macrophages was observed in Exo-srIκB-treated group, especially the hepatic frequencies of F4/80⁺Ly6C^{hi} pro-inflammatory macrophages were significantly decreased. In contrast, the frequencies of F4/80⁺Ly6C^{low} anti-inflammatory macrophages were increased in the Exo-srIκB-treated group, but Ly6G⁺CD11 b⁺ neutrophils or lymphocytes showed similar frequencies between the two groups (FIG. 16h).

### 3-6. Mitigation Effect of Alcoholic Liver Injury (ALI), Alcoholic Fatty Liver (AFL), and Alcoholic Liver Fibrosis (ALF) Medicated by Multiple Exo-srIκB Injections in Mice

The various therapeutic and preventative effects of Exo-siKB on alcoholic liver injury (ALI) were confirmed.

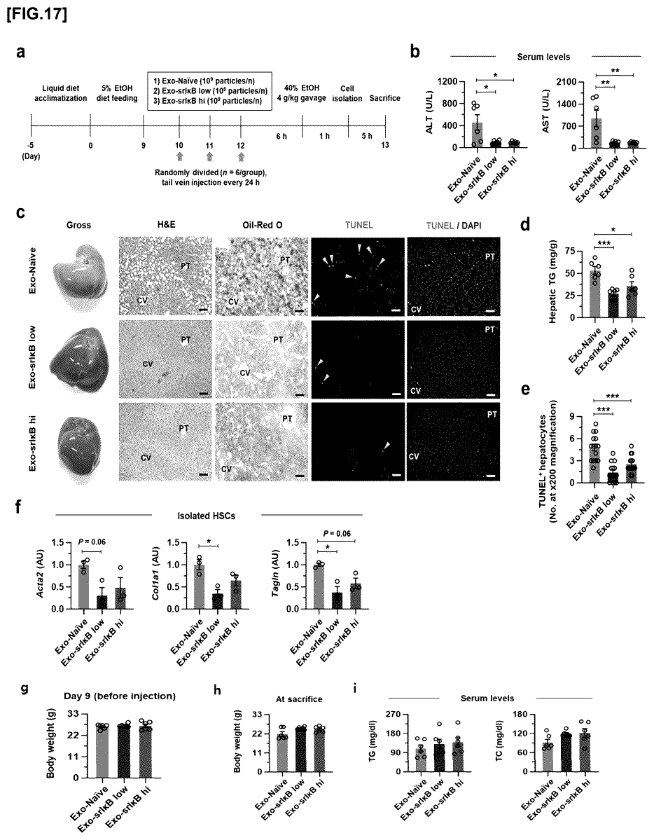

Specifically, WT mice were fed EtOH (4 g kg⁻¹ of 40% EtOH gavage) for 9 days and randomly divided them into 3 groups, which received 3 consecutive days of Exo-Naïve (10⁹ particles/day/mouse), low dose of Exo-srIκB (10⁸ particles/day/mouse), or high dose of Exo-srIκB (10⁹ particles/day/mouse) before alcohol drinking (FIG. 17a).

As a result, it was confirmed that the serum ALT and AST levels in both low-dose and high-dose Exo-srIκB injected mice were significantly reduced compared to those of control mice with similar body weight and serum TG and TC levels (FIG. 17b and FIG. 17g-i).

In addition, as a result of performing H&E staining and Oil Red O staining in order to confirm the total lipid content, it was confirmed that lipid accumulation in the midzonal apoptotic hepatocytes (HEPs) was markedly suppressed by Exo-srI_B treatment (FIG. 17c and FIG. 17d).

The terminal deoxynucleotidyl transferase dUTP nick end labeling (TUNEL) assay (Abcam, Cambridge, UK) was performed according to the manufacturers instructions in order to confirm whether apoptosis of HEPs and non-parenchymal cell of liver tissue was reduced, and images were captured using Olympus BX51 microscope equipped with a CCD camera (Olympus, Tokyo, Japan) and analyzed with DP2-BSW.

As a result, in the case of Exo-srIκB treatment, the apoptosis of HEP and non-parenchymal cell was diminished in the midzonal area of Exo-Naïve-treated mice (FIG. 17c and FIG. 17d).

Finally, in isolated hepatic stellate cell (HSC), expression levels of genes related to fibrotic activation (Acta2, Col1a1, and TagIn) were significantly reduced in the low-dose Exo-srIκB group and also decreased in the high-dose Exo-srIκB group compared to those of the control group (FIG. 17f).

### 3-7. Mitigation Effect of Alcoholic Liver Inflammation by Inhibition of KC Activity

To explore the underlying mechanisms of the protective effects of Exo-srIκB in ALD, the KC, the major target cell type for Exo-srIκB, were analyzed from the multiple Exo-srIκB injection animal model of Example 3-6. The KC were analyzed based on the known studies which have confirmed that, in the progression of non-alcoholic steatohepatitis, there is a gradual loss of embryonic-derived KC (emKC) and an eventual replacement by bone marrow-derived macrophages (bmKC) (Remmerie, A. et al., Immunity 2020, 53, 641-657; Bonnardel, J.; T'Jonck, W. et al., Immunity 2019, 51, 638-654.). In particular, CLEC2 and TIM4 were used as markers for emKC (CLEC2+TIM4+), which can distinguish them from bmKC (CLEC2+TIM4−), and flow cytometry was performed.

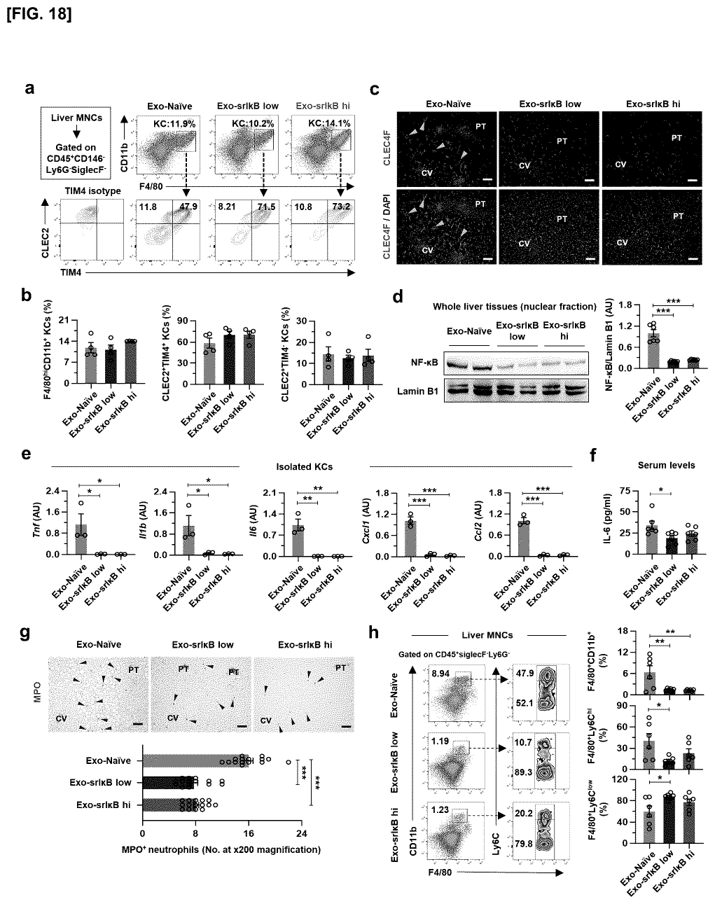

As a result, there were no differences in the frequencies of F4/80hiCD11b+ KC, emKC (CLEC2+TIM4+), and bmKC (CLEC2+TIM4−) between the three groups (FIG. 18a and FIG. 18b).

Next, for histological analyses, similar regions of the left and medial lobes of mouse liver were used. As a result, the immunostaining of liver tissues showed a similar number of CLEC4F (a marker for both emKC and bmKC)-expressing cell (FIG. 18c). However, in the liver tissues of Exo-Naïve-treated mice, morphological changes were found in the midzonal CLEC4F+ KC, which disappeared by Exo-srIκB treatment, suggesting functional alterations in KC by Exo-srIκB (FIG. 18c).

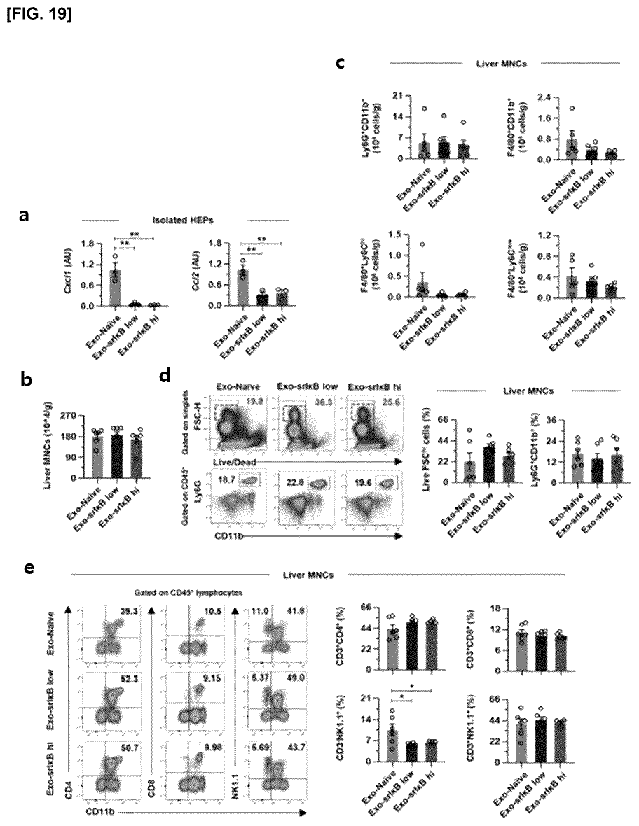

Additionally, it was confirmed that the nuclear translocation of NF-B and expression levels of pro-inflammatory genes (Tnf, Il1b, and Il6) in whole liver tissues or isolated KC from Exo-srIκB-treated mice were reduced compared to that of the control mice (FIG. 18d and FIG. 18e). It was also confirmed that the serum IL-6 levels in Exo-sdIκB-treated mice were decreased (FIG. 18f). In addition, expression levels of CXCL1 and CCL2 in isolated KC and HEPs from Exo-srIκB-treated mice were decreased compared to those of the control mice (FIGS. 18e and 19a).

In fact, the number of MPO+ neutrophils was significantly decreased in Exo-srIκB-treated mice in the intact liver tissue, especially in the midzonal area (FIG. 18g). Additionally, although the FSC^{hi} granulocytes such as Ly6G⁺CD11b⁺ neutrophils showed similar frequencies in all groups, diminished hepatic frequencies of F4/80+CD11 b macrophages, particularly F4/80⁺Ly6C^{hi} pro-inflammatory macrophages and increased frequencies of F4/80⁺Ly6C^{low} anti-inflammatory macrophages in Exo-srIκB-treated mice were confirmed (FIGS. 18h and 19d).

These results indicate that there are hepatoprotective effects of multiple Exo-srIκB injections in ALD mediated by suppression of inflammatory KC.

In summary, it was confirmed that the Exo-srIκB of the present invention can effectively inhibit lipid accumulation, apoptosis, acute inflammation and hepatic fibrosis of liver tissues upon administration, and based on this, it can be provided as an effective therapeutic agent for alcoholic liver disease in which the above symptoms appear in combination.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A method for treating liver disease, comprising administering to a subject in need thereof a pharmaceutical composition comprising an exosome containing super-repressor-IκB (srIκB) as an active ingredient.

2. The method of claim 1, wherein the liver disease is liver fibrosis, liver cirrhosis, fatty liver, alcoholic liver disease cholestasis, or a combination thereof.

3. The method of claim 2, wherein the alcoholic liver disease is alcoholic fatty liver, alcoholic steatohepatitis, alcoholic cirrhosis, alcoholic liver fibrosis, or a combination thereof.

4. The method of claim 1, wherein the composition is administered via an oral, transdermal, intraperitoneal, intravenous (IV), intramuscular, subcutaneous, or a combination thereof.

5. The method of claim 1, wherein the dosage of the exosome is from 1×10⁶ pn/kg to 1×10¹⁵ pn/kg.
